(19) United States
(12) Patent Application Publication
(10) Pub. No.: US 2012/0215122 A1
(43) Pub. Date: Aug. 23, 2012

(54) ARRHYTHMIA INFORMATION MANAGEMENT

(75) Inventor: Nassir F. MARROUCHE, Park City, UT (US)

(73) Assignee: University of Utah Research Foundation, Salt Lake City, UT (US)

(21) Appl. No.: 13/031,068

(22) Filed: Feb. 18, 2011

(51) Int. Cl. A61B 5/02

(52) U.S. Cl. 600/508

(57) ABSTRACT

Systems and methods for managing cardiac information concerning a patient are provided. In some aspects, cardiac information objects are displayed graphically at an interface having at least two axes. Time is displayed on a first axis of the at least two axes and parameters of the cardiac information objects are displayed on a second axis of the at least two axes. Each cardiac information object is spatially associated with at least a portion of the time displayed and a parameter displayed. In some aspects, the interface advantageously allows a user to conveniently view and manage the cardiac information objects.

### FIELD

The present invention generally relates to medical information management.

### BACKGROUND

While caring for patients, clinicians typically encounter and process various medical information concerning the patients. This medical information can be large in volume, especially when clinicians provide long-term care to patients or when clinicians provide care to many different patients. Managing this large amount of medical information can therefore be difficult.

### SUMMARY

According to various embodiments of the subject technology, systems and methods for managing medical information are provided. In some embodiments, medical information objects are displayed graphically at an interface having at least two axes. The interface advantageously allows a user to conveniently view and manage the medical information objects. Embodiments of the subject technology allow the user to access further detailed information associated with each medical information object displayed at the interface. This information may also be displayed at the interface.

According to various embodiments of the subject technology, a method for managing medical information of a patient is provided. The method comprises facilitating, by a processor, displaying medical information objects graphically at an interface having at least two axes. Time is displayed on a first axis of the at least two axes. Parameters of the medical information objects are displayed on a second axis of the at least two axes. Each medical information object is spatially associated with at least a portion of the time displayed and a parameter displayed. In some embodiments, the displayed parameters respectively comprise a medical imaging category, a measure of anticoagulation, a medical procedure category, an encounter of the patient with a clinician, a cardiac rhythm category, a patient portal, and a therapy category. The method also comprises receiving, by a processor, an inquiry input associated with a first of the medical information objects. The method also comprises facilitating displaying, in response to the inquiry input and by a processor, secondary medical information corresponding to the parameter spatially associated with the first of the medical information objects.

According to certain embodiments, when the parameter spatially associated with the first of the medical information objects is the medical imaging category, the secondary medical information comprises at least one of an image, a video, and an imaging report. In some embodiments, when the parameter spatially associated with the first of the medical information objects is the measure of anticoagulation, the secondary medical information comprises a value of the measure of anticoagulation. When the parameter spatially associated with the first of the medical information objects is the medical procedure category, the secondary medical information comprises at least one of an image, a video, and a report related to a procedure performed on the patient.

In some embodiments, when the parameter spatially associated with the first of the medical information objects is the encounter, the secondary medical information comprises a report of the encounter. When the parameter spatially associated with the first of the medical information objects is the cardiac rhythm category, the secondary medical information comprises at least one of an electrocardiogram and a report of an electrocardiogram. When the parameter spatially associated with the first of the medical information objects is the patient portal, the secondary medical information comprises a patient self-report of at least one of a symptom and a medication use.

According to certain embodiments, the medical information object spatially associated with the therapy category parameter comprises (a) a first edge indicating a time of onset of a prescribed therapy, and (b) a second edge indicating (i) a time of cessation of the prescribed therapy, or (ii) a present time. The prescribed therapy comprises administration of a medication, and at least one of a width and a color of the medical information objects associated with the prescribed therapy varies with a dosage of the medication.

In some embodiments, when the parameter spatially associated with the first of the medical information objects is the medical imaging category, the secondary medical information comprises at least one of an echocardiogram image, an echocardiogram video, and an echocardiogram report.

According to certain embodiments, the medical information objects are displayed discretely along the first axis. The method also comprises receiving input from the patient. The input received from the patient is configured to generate the patient self-report. In some embodiments, the method also comprises generating the patient self-report based on the input received from the patient.

In some embodiments, the measure of anticoagulation comprises a prothrombin time. The measure of anticoagulation comprises an international normalized ratio of prothrombin time. In some embodiments, the report of the encounter comprises at least one of a report of a patient history and report of a physical examination of the patient. In some embodiments, the first axis and the second axis are orthogonal to one another.

In some embodiments, the inquiry input comprises at least one of a mouse event and a keyboard event. The method also comprises facilitating expanding or facilitating compressing a representation of the first axis such that the medical information objects associated with the at least the portion of the time displayed on the first axis are correspondingly expanded or compressed graphically.

According to various embodiments of the subject technology, a non-transitory machine-readable medium encoded with instructions executable by a processing system to perform a method for managing medical information of a patient is provided. The instructions comprise code for facilitating, by a processor, displaying medical information objects graphically at an interface having at least two axes. Time is displayed on a first axis of the at least two axes. Parameters of the medical information objects are displayed on a second axis of the at least two axes. Each medical information object is spatially associated with at least a portion of the time displayed and a parameter displayed. In some embodiments, the displayed parameters respectively comprise a medical imaging category, a measure of anticoagulation, a medical procedure category, an encounter of the patient with a clinician, a cardiac rhythm category, a patient portal, and a therapy category. The instructions also comprise code for receiving, by a processor, an inquiry input associated with a first of the medical information objects. The instructions also comprise code for facilitating displaying, in response to the inquiry input and by a processor, secondary medical information corresponding to the parameter spatially associated with the first of the medical information objects.

According to certain embodiments, when the parameter spatially associated with the first of the medical information objects is the medical imaging category, the secondary medical information comprises at least one of an image, a video, and an imaging report. In some embodiments, when the parameter spatially associated with the first of the medical information objects is the measure of anticoagulation, the secondary medical information comprises a value of the measure of anticoagulation. When the parameter spatially associated with the first of the medical information objects is the medical procedure category, the secondary medical information comprises at least one of an image, a video, and a report related to a procedure performed on the patient.

In some embodiments, when the parameter spatially associated with the first of the medical information objects is the encounter, the secondary medical information comprises a report of the encounter. When the parameter spatially associated with the first of the medical information objects is the cardiac rhythm category, the secondary medical information comprises at least one of an electrocardiogram and a report of an electrocardiogram. When the parameter spatially associated with the first of the medical information objects is the patient portal, the secondary medical information comprises a patient self-report of at least one of a symptom and a medication use.

According to certain embodiments, the medical information object spatially associated with the therapy category parameter comprises (a) a first edge indicating a time of onset of a prescribed therapy, and (b) a second edge indicating (i) a time of cessation of the prescribed therapy, or (ii) a present time. The prescribed therapy comprises administration of a medication, and at least one of a width and a color of the medical information objects associated with the prescribed therapy varies with a dosage of the medication.

In some embodiments, when the parameter spatially associated with the first of the medical information objects is the medical imaging category, the secondary medical information comprises at least one of an echocardiogram image, an echocardiogram video, and an echocardiogram report.

According to certain embodiments, the medical information objects are displayed discretely along the first axis. The instructions further comprise code for receiving input from the patient. The input received from the patient is configured to generate the patient self-report. In some embodiments, the instructions further comprise code for generating the patient self-report based on the input received from the patient.

In some embodiments, the measure of anticoagulation comprises a prothrombin time. The measure of anticoagulation comprises an international normalized ratio of prothrombin time. In some embodiments, the report of the encounter comprises at least one of a report of a patient history and report of a physical examination of the patient. In some embodiments, the first axis and the second axis are orthogonal to one another.

In some embodiments, the inquiry input comprises at least one of a mouse event and a keyboard event. The instructions further comprise code for facilitating expanding or facilitating compressing a representation of the first axis such that the medical information objects associated with the at least the portion of the time displayed on the first axis are correspondingly expanded or compressed graphically. In some embodiments, a computing machine comprises the machine-readable medium. In some embodiments, the computing machine comprises a server.

According to various embodiments of the subject technology, a system for managing medical information of a patient is provided. The system comprises a first display module configured to facilitate, by a processor, displaying medical information objects graphically at an interface having at least two axes. Time is displayed on a first axis of the at least two axes. Parameters of the medical information objects are displayed on a second axis of the at least two axes. Each medical information object is spatially associated with at least a portion of the time displayed and a parameter displayed. In some embodiments, the displayed parameters respectively comprise a medical imaging category, a measure of anticoagulation, a medical procedure category, an encounter of the patient with a clinician, a cardiac rhythm category, a patient portal, and a therapy category. The system also comprises a user input module configured to receive, by a processor, an inquiry input associated with a first of the medical information objects. The system also comprises a second display module configured to facilitate displaying, in response to the inquiry input and by a processor, secondary medical information corresponding to the parameter spatially associated with the first of the medical information objects.

According to certain embodiments, when the parameter spatially associated with the first of the medical information objects is the medical imaging category, the secondary medical information comprises at least one of an image, a video, and an imaging report. In some embodiments, when the parameter spatially associated with the first of the medical information objects is the measure of anticoagulation, the secondary medical information comprises a value of the measure of anticoagulation. When the parameter spatially associated with the first of the medical information objects is the medical procedure category, the secondary medical information comprises at least one of an image, a video, and a report related to a procedure performed on the patient.

In some embodiments, when the parameter spatially associated with the first of the medical information objects is the encounter, the secondary medical information comprises a report of the encounter. When the parameter spatially associated with the first of the medical information objects is the cardiac rhythm category, the secondary medical information comprises at least one of an electrocardiogram and a report of an electrocardiogram. When the parameter spatially associated with the first of the medical information objects is the patient portal, the secondary medical information comprises a patient self-report of at least one of a symptom and a medication use.

According to certain embodiments, the medical information object spatially associated with the therapy category parameter comprises (a) a first edge indicating a time of onset of a prescribed therapy, and (b) a second edge indicating (i) a time of cessation of the prescribed therapy, or (ii) a present time. The prescribed therapy comprises administration of a medication, and at least one of a width and a color of the medical information objects associated with the prescribed therapy varies with a dosage of the medication.

In some embodiments, when the parameter spatially associated with the first of the medical information objects is the medical imaging category, the secondary medical information comprises at least one of an echocardiogram image, an echocardiogram video, and an echocardiogram report.

According to certain embodiments, the medical information objects are displayed discretely along the first axis. The system also comprises a patient input module configured to receive input from the patient. The input received from the patient is configured to generate the patient self-report. In some embodiments, the patient input module is configured to generate the patient self-report based on the input received from the patient.

In some embodiments, the measure of anticoagulation comprises a prothrombin time. The measure of anticoagulation comprises an international normalized ratio of prothrombin time. In some embodiments, the report of the encounter comprises at least one of a report of a patient history and report of a physical examination of the patient. In some embodiments, the first axis and the second axis are orthogonal to one another.

In some embodiments, the inquiry input comprises at least one of a mouse event and a keyboard event. The system also comprises an adjustment module configured to facilitate expanding or facilitate compressing a representation of the first axis such that the medical information objects associated with the at least the portion of the time displayed on the first axis are correspondingly expanded or compressed graphically. In some embodiments, the system comprises a server. In some embodiments, the system comprises a processor and a memory.

According to various embodiments of the subject technology, a method for managing cardiac arrhythmia information concerning a patient is provided. The method comprises facilitating, by a processor, displaying cardiac information objects graphically at an interface having at least two axes. Time is displayed on a first axis of the at least two axes. Parameters of the cardiac information objects are displayed on a second axis of the at least two axes. Each cardiac information object is spatially associated with at least a portion of the time displayed and a parameter displayed. In some embodiments, the displayed parameters respectively comprise a cardiac imaging category, a measure of anticoagulation, a cardiac procedure category, an encounter of the patient with a clinician, a cardiac rhythm category, a patient portal, and a cardiac therapy category. The method also comprises receiving, by a processor, an inquiry input associated with a first of the cardiac information objects. The method also comprises facilitating displaying, in response to the inquiry input and by a processor, secondary cardiac information corresponding to the parameter spatially associated with the first of the cardiac information objects.

According to certain embodiments, when the parameter spatially associated with the first of the cardiac information objects is the cardiac imaging category, the secondary cardiac information comprises at least one of an image, a video, and an imaging report. In some embodiments, when the parameter spatially associated with the first of the cardiac information objects is the measure of anticoagulation, the secondary cardiac information comprises a value of the measure of anticoagulation. When the parameter spatially associated with the first of the cardiac information objects is the cardiac procedure category, the secondary cardiac information comprises at least one of an image, a video, and a report related to a cardiac procedure performed on the patient.

In some embodiments, when the parameter spatially associated with the first of the cardiac information objects is the encounter, the secondary cardiac information comprises a report of the encounter. When the parameter spatially associated with the first of the cardiac information objects is the cardiac rhythm category, the secondary cardiac information comprises at least one of an electrocardiogram and a report of an electrocardiogram. When the parameter spatially associated with the first of the cardiac information objects is the patient portal, the secondary cardiac information comprises a patient self-report of at least one of a symptom and a medication use.

According to certain embodiments, the cardiac information object spatially associated with the cardiac therapy category parameter comprises (a) a first edge indicating a time of onset of a prescribed therapy, and (b) a second edge indicating (i) a time of cessation of the prescribed therapy, or (ii) a present time. The prescribed therapy comprises administration of a medication, and wherein at least one of a width and a color of the cardiac information objects associated with the prescribed therapy varies with a dosage of the medication.

In some embodiments, when the parameter spatially associated with the first of the cardiac information objects is the cardiac imaging category, the secondary cardiac information comprises at least one of an echocardiogram image, an echocardiogram video, and an echocardiogram report.

According to certain embodiments, the cardiac information objects are displayed discretely along the first axis. The method also comprises receiving input from the patient. The input received from the patient is configured to generate the patient self-report. In some embodiments, the method also comprises generating the patient self-report based on the input received from the patient.

In some embodiments, wherein the measure of anticoagulation comprises a prothrombin time. The measure of anticoagulation comprises an international normalized ratio of prothrombin time. In some embodiments, the report of the encounter comprises at least one of a report of a patient history and report of a physical examination of the patient. In some embodiments, the first axis and the second axis are orthogonal to one another.

In some embodiments, the inquiry input comprises at least one of a mouse event and a keyboard event. The method also comprises facilitating expanding or facilitating compressing a representation of the first axis such that the cardiac information objects associated with the at least the portion of the time displayed on the first axis are correspondingly expanded or compressed graphically.

According to various embodiments of the subject technology, a non-transitory machine-readable medium encoded with instructions executable by a processing system to perform a method for managing cardiac arrhythmia information concerning a patient is provided. The instructions comprise code for facilitating, by a processor, displaying cardiac information objects graphically at an interface having at least two axes. Time is displayed on a first axis of the at least two axes. Parameters of the cardiac information objects are displayed on a second axis of the at least two axes. Each cardiac information object is spatially associated with at least a portion of the time displayed and a parameter displayed. In some embodiments, the displayed parameters respectively comprise a cardiac imaging category, a measure of anticoagulation, a cardiac procedure category, an encounter of the patient with a clinician, a cardiac rhythm category, a patient portal, and a cardiac therapy category. The instructions also comprise code for receiving, by a processor, an inquiry input associated with a first of the cardiac information objects. The instructions also comprise code for facilitating displaying, in response to the inquiry input and by a processor, secondary cardiac information corresponding to the parameter spatially associated with the first of the cardiac information objects.

According to certain embodiments, when the parameter spatially associated with the first of the cardiac information objects is the cardiac imaging category, the secondary cardiac information comprises at least one of an image, a video, and an imaging report. In some embodiments, when the parameter spatially associated with the first of the cardiac information objects is the measure of anticoagulation, the secondary cardiac information comprises a value of the measure of anticoagulation. When the parameter spatially associated with the first of the cardiac information objects is the cardiac procedure category, the secondary cardiac information comprises at least one of an image, a video, and a report related to a cardiac procedure performed on the patient.

In some embodiments, when the parameter spatially associated with the first of the cardiac information objects is the encounter, the secondary cardiac information comprises a report of the encounter. When the parameter spatially associated with the first of the cardiac information objects is the cardiac rhythm category, the secondary cardiac information comprises at least one of an electrocardiogram and a report of an electrocardiogram. When the parameter spatially associated with the first of the cardiac information objects is the patient portal, the secondary cardiac information comprises a patient self-report of at least one of a symptom and a medication use.

According to certain embodiments, the cardiac information object spatially associated with the cardiac therapy category parameter comprises (a) a first edge indicating a time of onset of a prescribed therapy, and (b) a second edge indicating (i) a time of cessation of the prescribed therapy, or (ii) a present time. The prescribed therapy comprises administration of a medication, and wherein at least one of a width and a color of the cardiac information objects associated with the prescribed therapy varies with a dosage of the medication.

In some embodiments, when the parameter spatially associated with the first of the cardiac information objects is the cardiac imaging category, the secondary cardiac information comprises at least one of an echocardiogram image, an echocardiogram video, and an echocardiogram report.

According to certain embodiments, the cardiac information objects are displayed discretely along the first axis. The instructions also comprise code for receiving input from the patient. The input received from the patient is configured to generate the patient self-report. In some embodiments, the instructions also comprise code for generating the patient self-report based on the input received from the patient.

In some embodiments, wherein the measure of anticoagulation comprises a prothrombin time. The measure of anticoagulation comprises an international normalized ratio of prothrombin time. In some embodiments, the report of the encounter comprises at least one of a report of a patient history and report of a physical examination of the patient. In some embodiments, the first axis and the second axis are orthogonal to one another.

In some embodiments, the inquiry input comprises at least one of a mouse event and a keyboard event. The instructions also comprise code for facilitating expanding or facilitating compressing a representation of the first axis such that the cardiac information objects associated with the at least the portion of the time displayed on the first axis are correspondingly expanded or compressed graphically. In some embodiments, a computing machine comprises the machine-readable medium. In some embodiments, the computing machine comprises a server.

According to various embodiments of the subject technology, a system for managing cardiac arrhythmia information concerning a patient is provided. The system comprises a first display module configured to facilitate, by a processor, displaying cardiac information objects graphically at an interface having at least two axes. Time is displayed on a first axis of the at least two axes. Parameters of the cardiac information objects are displayed on a second axis of the at least two axes. Each cardiac information object is spatially associated with at least a portion of the time displayed and a parameter displayed. In some embodiments, the displayed parameters respectively comprise a cardiac imaging category, a measure of anticoagulation, a cardiac procedure category, an encounter of the patient with a clinician, a cardiac rhythm category, a patient portal, and a cardiac therapy category. The system also comprises a user input module configured to receive, by a processor, an inquiry input associated with a first of the cardiac information objects. The system also comprises a second display module configured to facilitate displaying, in response to the inquiry input and by a processor, secondary cardiac information corresponding to the parameter spatially associated with the first of the cardiac information objects.

According to certain embodiments, when the parameter spatially associated with the first of the cardiac information objects is the cardiac imaging category, the secondary cardiac information comprises at least one of an image, a video, and an imaging report. In some embodiments, when the parameter spatially associated with the first of the cardiac information objects is the measure of anticoagulation, the secondary cardiac information comprises a value of the measure of anticoagulation. When the parameter spatially associated with the first of the cardiac information objects is the cardiac procedure category, the secondary cardiac information comprises at least one of an image, a video, and a report related to a cardiac procedure performed on the patient.

In some embodiments, when the parameter spatially associated with the first of the cardiac information objects is the encounter, the secondary cardiac information comprises a report of the encounter. When the parameter spatially associated with the first of the cardiac information objects is the cardiac rhythm category, the secondary cardiac information comprises at least one of an electrocardiogram and a report of an electrocardiogram. When the parameter spatially associated with the first of the cardiac information objects is the patient portal, the secondary cardiac information comprises a patient self-report of at least one of a symptom and a medication use.

According to certain embodiments, the cardiac information object spatially associated with the cardiac therapy category parameter comprises (a) a first edge indicating a time of onset of a prescribed therapy, and (b) a second edge indicating (i) a time of cessation of the prescribed therapy, or (ii) a present time. The prescribed therapy comprises administration of a medication, and wherein at least one of a width and a color of the cardiac information objects associated with the prescribed therapy varies with a dosage of the medication.

In some embodiments, when the parameter spatially associated with the first of the cardiac information objects is the cardiac imaging category, the secondary cardiac information comprises at least one of an echocardiogram image, an echocardiogram video, and an echocardiogram report.

According to certain embodiments, the cardiac information objects are displayed discretely along the first axis. The system also comprises a patient input module configured to receive input from the patient. The input received from the patient is configured to generate the patient self-report. In some embodiments, the patient input module is configured to generate the patient self-report based on the input received from the patient.

In some embodiments, wherein the measure of anticoagulation comprises a prothrombin time. The measure of anticoagulation comprises an international normalized ratio of prothrombin time. In some embodiments, the report of the encounter comprises at least one of a report of a patient history and report of a physical examination of the patient. In some embodiments, the first axis and the second axis are orthogonal to one another.

In some embodiments, the inquiry input comprises at least one of a mouse event and a keyboard event. The system also comprises an adjustment module configured to facilitate expanding or facilitate compressing a representation of the first axis such that the cardiac information objects associated with the at least the portion of the time displayed on the first axis are correspondingly expanded or compressed graphically. In some embodiments, the system comprises a server. In some embodiments, the system comprises a processor and a memory.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 illustrates a simplified diagram of a system, in accordance with various embodiments of the subject technology.

FIG. 2 illustrates a simplified block diagram of a server, in accordance with various embodiments of the subject technology.

FIG. 3 illustrates an example of a method for managing medical information of a patient, in accordance with various embodiments of the subject technology.

FIGS. 4A, 4B, 4C, 4D, and 4E illustrate examples of an interface, in accordance with various embodiments of the subject technology.

FIGS. 5A and 5B illustrate examples of an interface used for collecting information from a patient, in accordance with various embodiments of the subject technology.

FIG. 6 is a conceptual block diagram illustrating an example of a system, in accordance with various embodiments of the subject technology.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

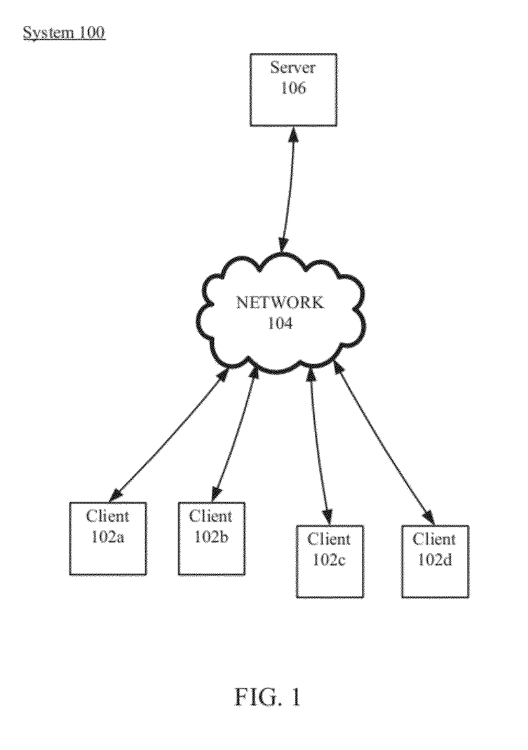

FIG. 1 illustrates a simplified diagram of a system 100, in accordance with various embodiments of the subject technology. The system 100 may include one ore more remote client devices 102 (e.g., client devices 102a, 102b, 102c, and 102d) in communication with a server computing device 106 (server) via a network 104. In some embodiments, the server 106 is configured to run applications that may be accessed and controlled at the client devices 102. For example, a user at a client device 102 may use a web browser to access and control an application running on the server 106 over the network 104. In some embodiments, the server 106 is configured to allow remote sessions (e.g., remote desktop sessions) wherein users can access applications and files on the server 106 by logging onto the server 106 from a client device 102. Such a connection may be established using any of several well-known techniques such as the Remote Desktop Protocol (RDP) on a Windows-based server.

By way of illustration and not limitation, in one aspect of the disclosure, stated from a perspective of a server side (treating a server as a local device and treating a client device as a remote device), a server application is executed (or runs) at a server 106. While a remote client device 102 may receive and display a view of the server application on a display local to the remote client device 102, the remote client device 102 does not execute (or run) the server application at the remote client device 102. Stated in another way from a perspective of the client side (treating a server as remote device and treating a client device as a local device), a remote application is executed (or runs) at a remote server 106.

By way of illustration and not limitation, a client device 102 can represent a computer, a mobile phone, a laptop computer, a thin client device, a personal digital assistant (PDA), a portable computing device, or a suitable device with a processor. In one example, a client device 102 is a smartphone (e.g., iPhone, Android phone, Blackberry, etc.). In certain configurations, a client device 102 can represent an audio player, a game console, a camera, a camcorder, an audio device, a video device, a multimedia device, or a device capable of supporting a connection to a remote server. In one example, a client device 102 can be mobile. In another example, a client device 102 can be stationary. According to one aspect of the disclosure, a client device 102 may be a device having at least a processor and memory, where the total amount of memory of the client device 102 could be less than the total amount of memory in a server 106. In one example, a client device 102 does not have a hard disk. In one aspect, a client device 102 has a display smaller than a display supported by a server 106. In one aspect, a client device may include one or more client devices.

In some embodiments, a server 106 may represent a computer, a laptop computer, a computing device, a virtual machine (e.g., VMware® Virtual Machine), a desktop session (e.g., Microsoft Terminal Server), a published application (e.g., Microsoft Terminal Server) or a suitable device with a processor. In some embodiments, a server 106 can be stationary. In some embodiments, a server 106 can be mobile. In certain configurations, a server 106 may be any device that can represent a client device. In some embodiments, a server 106 may include one or more servers.

In one example, a first device is remote to a second device when the first device is not directly connected to the second device. In one example, a first remote device may be connected to a second device over a communication network such as a Local Area Network (LAN), a Wide Area Network (WAN), and/or other network.

When a client device 102 and a server 106 are remote with respect to each other, a client device 102 may connect to a server 106 over a network 104, for example, via a modem connection, a LAN connection including the Ethernet or a broadband WAN connection including DSL, Cable, T1, T3, Fiber Optics, Wi-Fi, or a mobile network connection including GSM, GPRS, 3G, WiMax or other network connection. A network 104 can be a LAN network, a WAN network, a wireless network, the Internet, an intranet or other network. A network 104 may include one or more routers for routing data between client devices and/or servers. A remote device (e.g., client device, server) on a network may be addressed by a corresponding network address, such as, but not limited to, an Internet protocol (IP) address, an Internet name, a Windows Internet name service (WINS) name, a domain name or other system name. These illustrate some examples as to how one device may be remote to another device. But the subject technology is not limited to these examples.

According to certain embodiments of the subject technology, the terms “server” and “remote server” are generally used synonymously in relation to a client device, and the word “remote” may indicate that a server is in communication with other device(s), for example, over a network connection(s).

According to certain embodiments of the subject technology, the terms “client device” and “remote client device” are generally used synonymously in relation to a server, and the word “remote” may indicate that a client device is in communication with a server(s), for example, over a network connection(s).

In some embodiments, a “client device” may be sometimes referred to as a client or vice versa. Similarly, a “server” may be sometimes referred to as a server device or vice versa.

In some embodiments, the terms “local” and “remote” are relative terms, and a client device may be referred to as a local client device or a remote client device, depending on whether a client device is described from a client side or from a server side, respectively. Similarly, a server may be referred to as a local server or a remote server, depending on whether a server is described from a server side or from a client side, respectively. Furthermore, an application running on a server may be referred to as a local application, if described from a server side, and may be referred to as a remote application, if described from a client side.

In some embodiments, devices placed on a client side (e.g., devices connected directly to a client device(s) or to one another using wires or wirelessly) may be referred to as local devices with respect to a client device and remote devices with respect to a server. Similarly, devices placed on a server side (e.g., devices connected directly to a server(s) or to one another using wires or wirelessly) may be referred to as local devices with respect to a server and remote devices with respect to a client device.

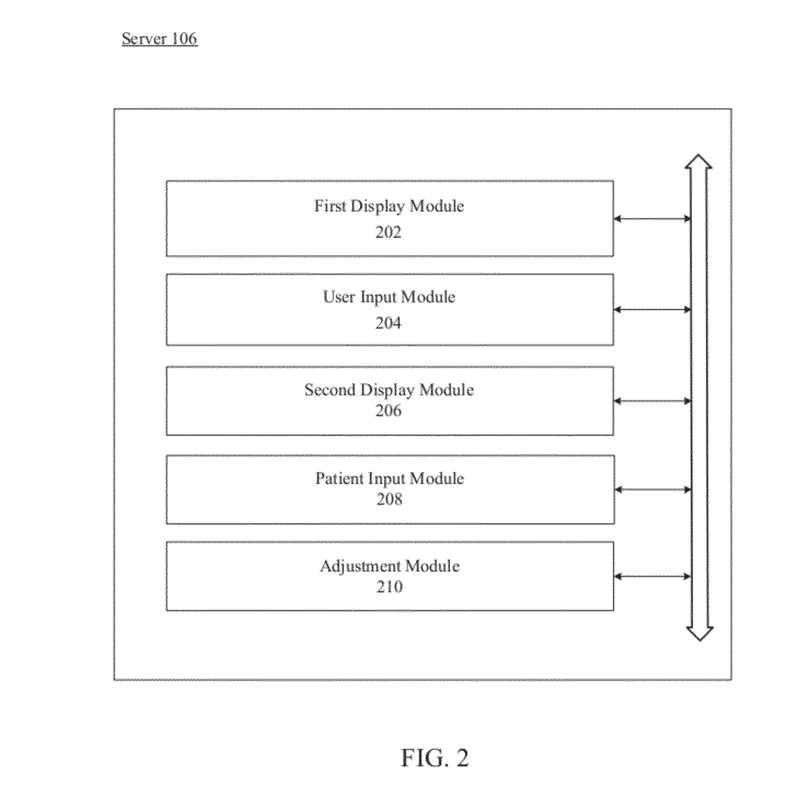

FIG. 2 illustrates a simplified block diagram of a server 106, in accordance with various embodiments of the subject technology. The server 106 comprises a first display module 202, a user input module 204, a second display module 206, a patient input module 208, and an adjustment module 210. In some embodiments, the server 106 is communicatively coupled with the network 104 via a network interface. The modules can be implemented in software, hardware and/or a combination of both. Features and functions of these modules according to various aspects are further described in the present disclosure.

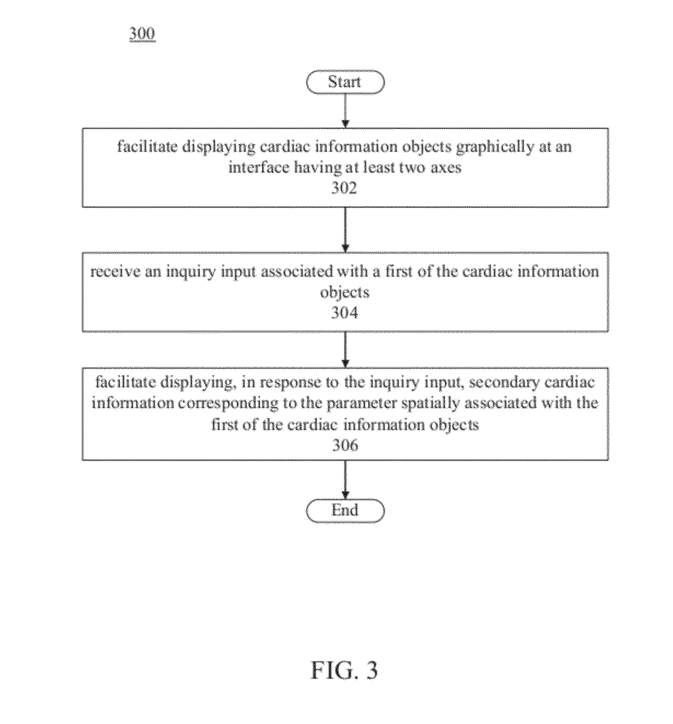

FIG. 3 illustrates an example of a method for managing medical information of a patient, in accordance with various embodiments of the subject technology. In particular, FIG. 3 illustrates a method 300 for managing cardiac arrhythmia information concerning a patient, in accordance with various embodiments of the subject technology. However, the subject technology is not limited to the management of cardiac arrhythmia information. In some embodiments, the subject technology may be applied for managing other suitable medical information including, for example and without limitation, information pertaining to cardiology, pulmonology, renalogy, radiology neurology, urology, orthopedics, psychiatry, dermatology, otorhinolaryngology (ear, nose, and throat), infectious diseases, internal medicine, gastroenterology, hepatology, and other medical fields. The method 300, for example, may be executed by and/or at the server 106. A user of the client device 102 may access the server 106, over the network 104, to utilize the method 300 for managing medical information. In some embodiments, the user of the client device 102 may be a clinician such a doctor, a nurse, or another suitable health care provider. In some embodiments, the user of the client device 102 may be the patient. Although the subject technology is described with respect to being implemented over the network 104, the subject technology may also be implemented by a standalone device for managing medical information.

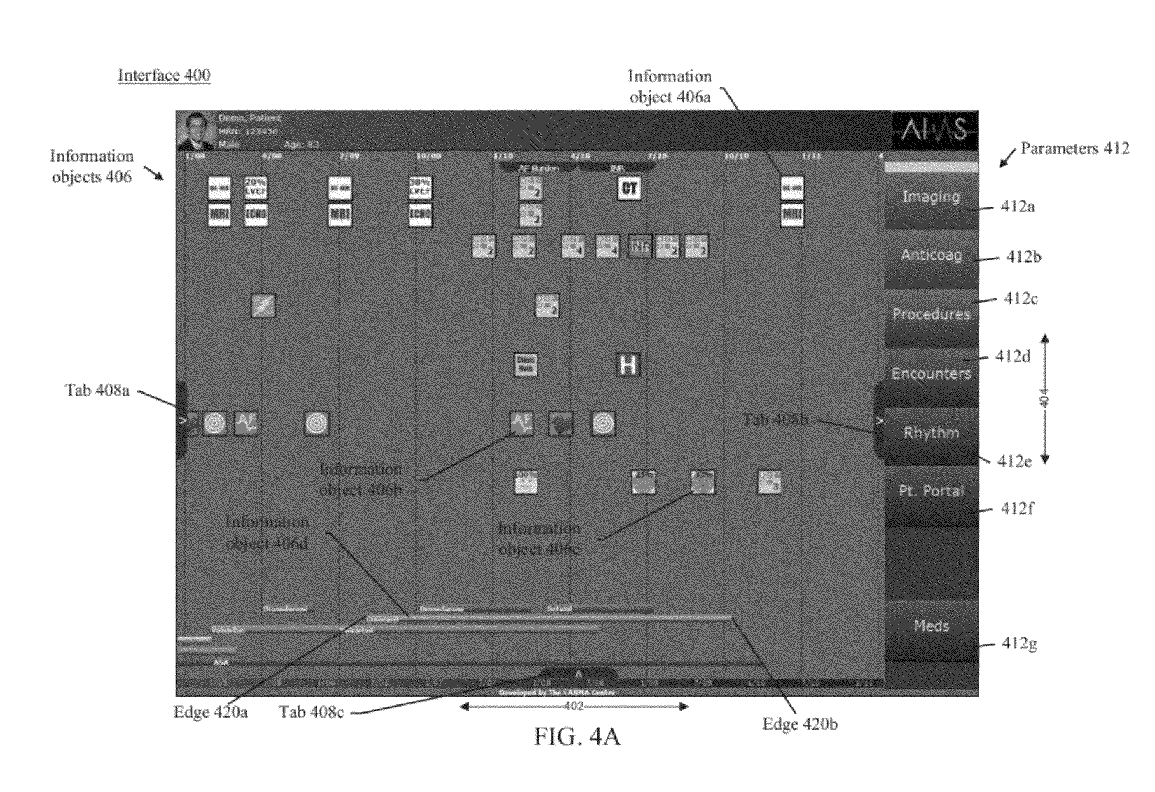

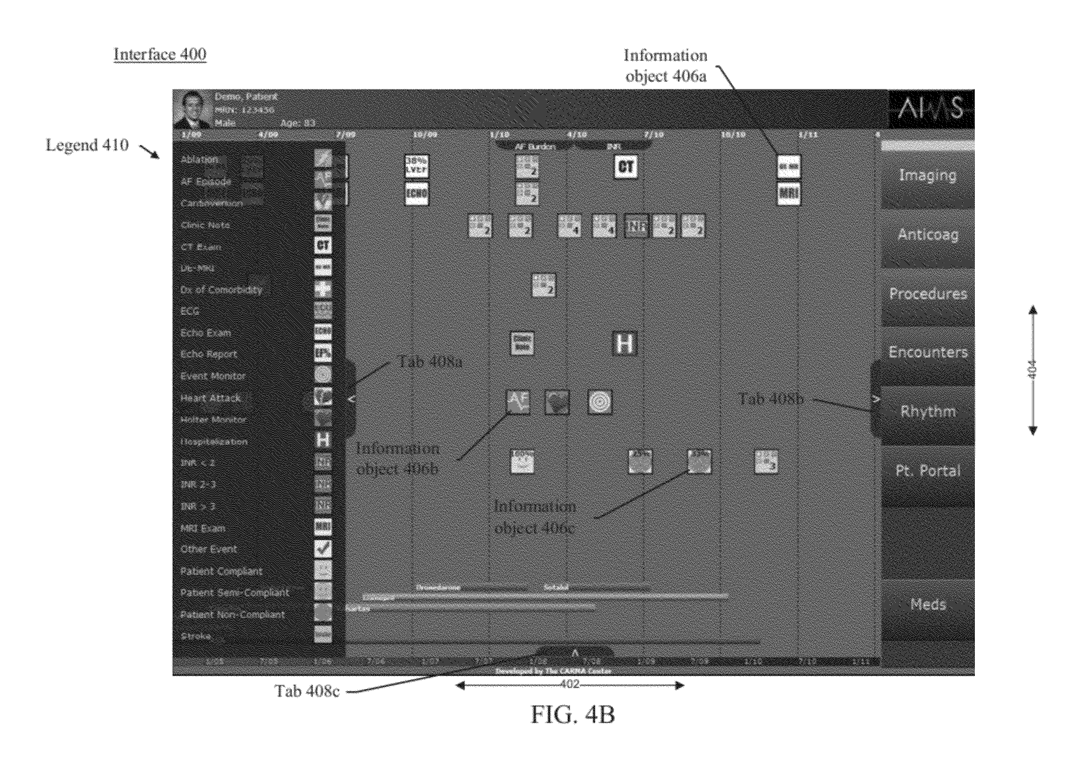

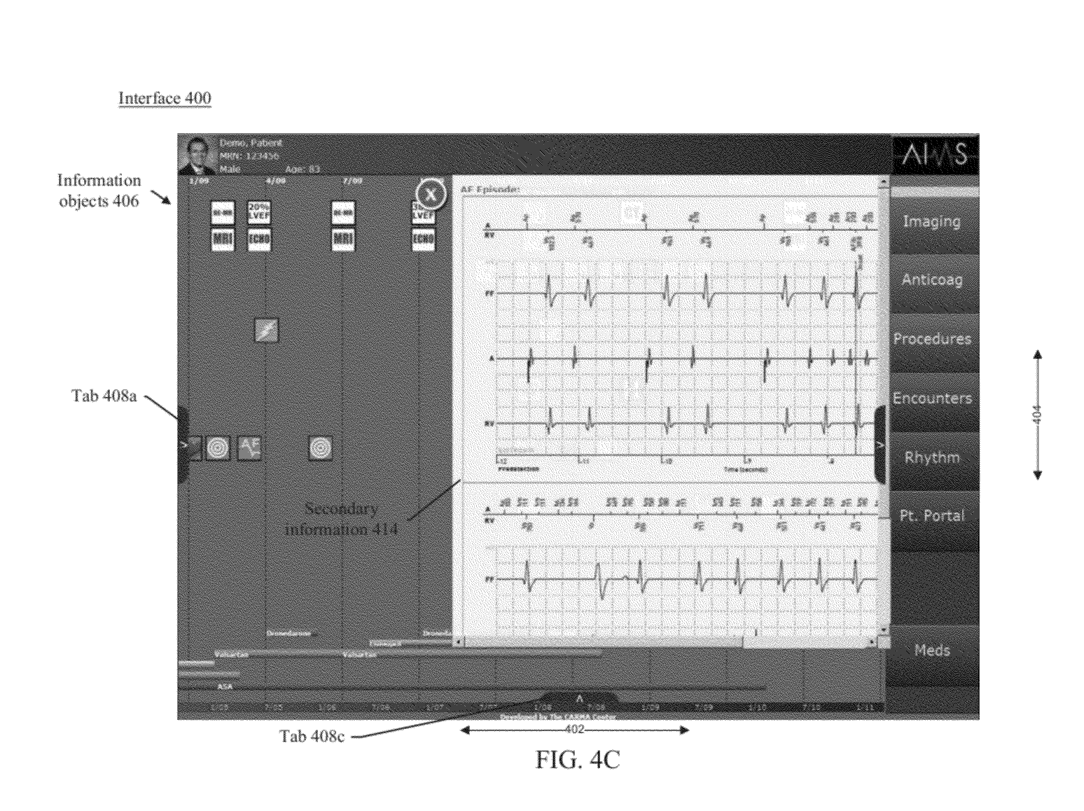

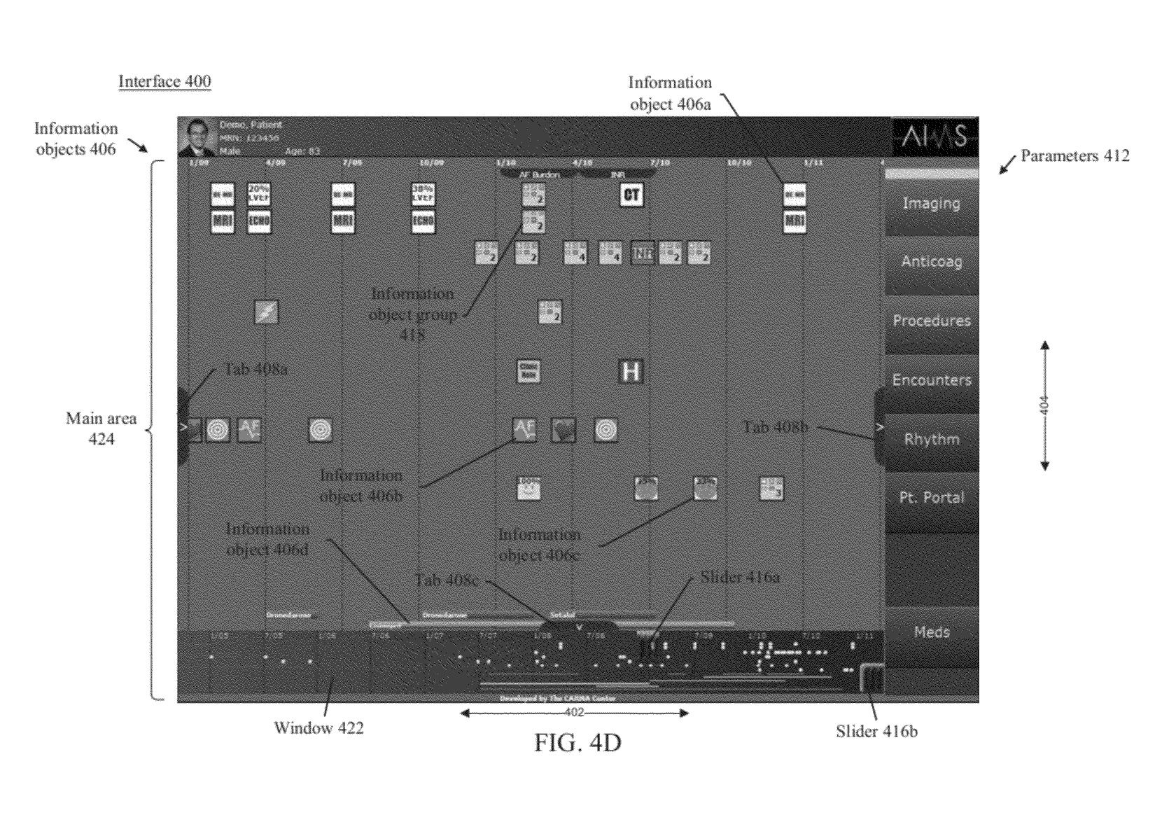

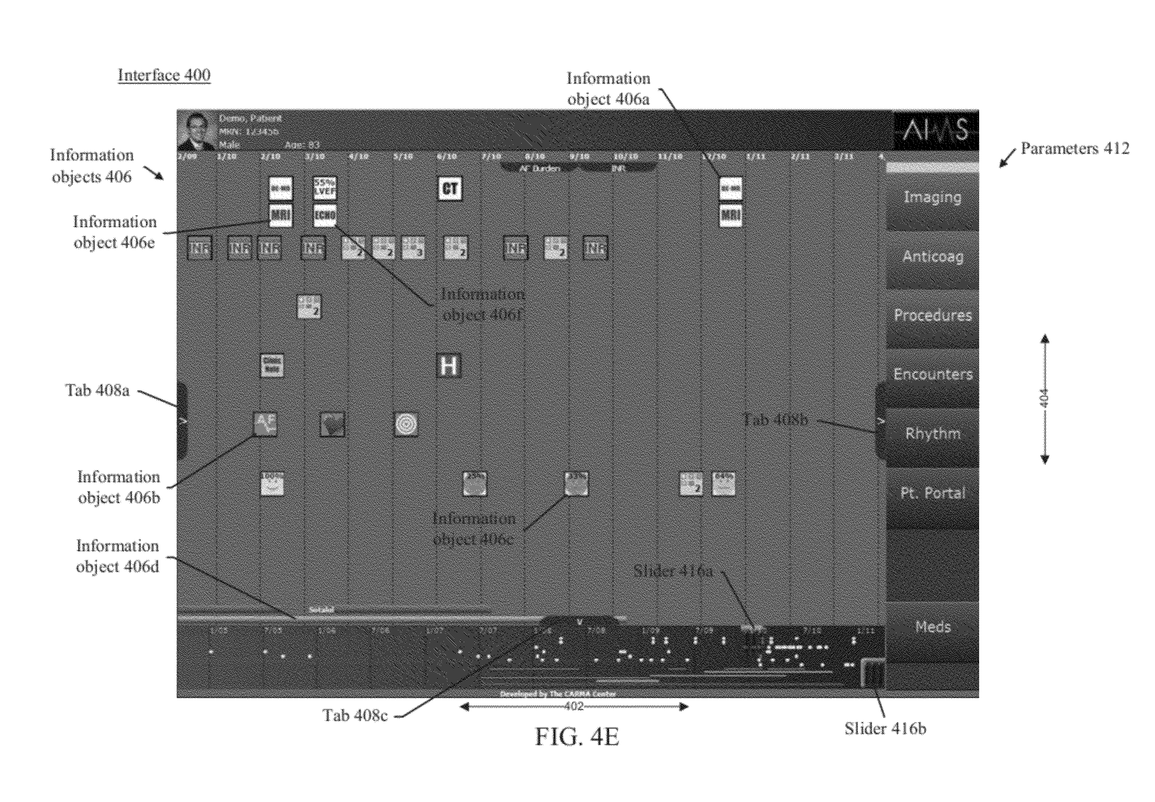

As shown in FIG. 3, the method 300 comprises facilitating displaying cardiac information objects graphically at an interface having at least two axes (e.g., 302). FIG. 4A illustrates an example of an interface 400 used for displaying medical information such as the cardiac information objects 406 (e.g., 406a, 406b, 406c, 406d, etc.), in accordance with various embodiments of the subject technology. For example, the interface 400 may be displayed at the client device 102. The user of the client device 102 may access the server 106 over the network 104 using a web browser (e.g., Internet Explorer, Firefox, Chrome, Safari, etc.) to receive instructions for displaying the interface 400 (e.g., on the web browser). For example, the first display module 202 may be configured to facilitate displaying the cardiac information objects 406 by transmitting, over the network 104, instructions and/or display data to client device 102. In response, the client device 102 may display the cardiac information objects 406 at the interface 400 based on the instructions and/or display data. In some embodiments, the instructions and/or display data comprises drawing commands for drawing the interface 400 and/or its associated components displayed on the interface 400 (e.g., the cardiac information objects 406).

As shown in FIG. 4A, the interface 400 includes axis 402 and axis 404. Time is displayed on the axis 402 and parameters 412 (e.g., 412a, 412b, 412c, 412d, 412e, 412f, 412g, etc.) of the cardiac information objects 406 are displayed on the axis 404. The axis 402 is orthogonal to axis 404. In some embodiments, each cardiac information object 406 is spatially associated with at least a portion of the time displayed and a parameter 412 displayed. In some embodiments, the displayed parameters 412 respectively comprise a cardiac imaging category (e.g., 412a), a measure of anticoagulation (e.g., 412b), a cardiac procedure category (e.g., 412c), an encounter of the patient with a clinician (e.g., 412d), a cardiac rhythm category (e.g., 412e), a patient portal (e.g., 412f), and a cardiac therapy category (e.g., 412g). The subject technology is not limited to these parameters and may include other suitable parameters such as parameters related to other medical fields. In some embodiments, a user may add or subtract parameters 412 displayed on the interface 400 depending what is desired to be displayed for managing medical information concerning a patient.

In some embodiments, the interface includes tabs 408 (e.g., 408a, 408b, 408c, etc.). In one example, the user of the client device 102 may click on the tab 408b to hide the parameters 412 from view. The tab 408b may be clicked again to display the parameters 412.

As shown in FIG. 4A, the cardiac information objects 406 are displayed discretely along the axis 402. Each of the cardiac information objects 406 may represent different information. In some embodiments, by activating the tab 408a, a legend may be displayed. The legend can provide an explanation for the different symbols used to illustrate the cardiac information objects 406. FIG. 4B illustrates an example of a legend 410 of the interface 400, in accordance with various embodiments of the subject technology. The legend 410 may be displayed, for example, once the user of the client device 102 clicks on the tab 408a. As shown, the legend 410 provides an explanation for the different symbols used to illustrate the cardiac information objects 406. For example, the information object 406a refers to information related to delayed enhancement-magnetic resonance imaging (DE-MRI). The information object 406b refers to information related to an atrial fibrillation (AF) episode. The information object 406c refers to information related to an indicator of a patient being non-compliant (e.g., with taking prescribed therapies). The subject technology is not limited to the information illustrated by the information objects 406 in FIGS. 4A and 4B, but may include other types of suitable medical information. In some embodiments, a user may add or subtract information objects 406 displayed on the interface 400 depending what is desired to be displayed for managing medical information concerning a patient.

Returning to FIG. 4A, the cardiac information objects 406 may also refer to information related to the type and/or length of a prescribed therapy. For example, the information object 406d is spatially associated with the cardiac therapy category parameter 412g (e.g., medication). Thus, the information object 406d may refer to information related to the type of medicine associated with a particular patient, and the length of time that the patient has taken the medicine (e.g., as shown by the length of the information object 406d relative to the time axis 402). In some embodiments, the information object 406d comprises (a) a first edge 420a indicating a time of onset of a prescribed therapy, and (b) a second edge 420b indicating (i) a time of cessation of the prescribed therapy, or (ii) a present time. In some embodiments, the present time may refer to a time that a user of the client device 102 uses the interface 400 for managing medical information (e.g., a current time). In some embodiments, the prescribed therapy comprises administration of a medication. In some embodiments, at least one of a width and a color of the cardiac information object 406 associated with the prescribed therapy may vary with a dosage of the medication. For example, the color of the information object 406d may indicate that it is a different medication from other colors. In another example, the wider the information object 406d, the larger the dose of the medication represented by the information object 406d, and vice versa.

Returning to FIG. 3, the method 300 comprises receiving an inquiry input associated with a first of the cardiac information objects (e.g., 304). For example, turning to FIG. 4A, a user of the client device 102 may want to inquire about further details related to information object 406b. The user can click on the information object 406b (e.g., an example of an inquiry input), and the client device 102 may transmit this inquiry input to the server 106. In some embodiments, the inquiry input comprises at least one of a mouse event (e.g., a click), a keyboard event, and some other suitable event for interfacing a user with the client device 102. The user input module 204 can be configured to receive the inquiry input associated with the information object 406b.

The method 300 also comprises facilitating displaying, in response to the inquiry input, secondary cardiac information corresponding to the parameter spatially associated with the first of the cardiac information objects (e.g., 306). For example, the second display module 206 can be configured to facilitate displaying, in response to the inquiry input, the secondary cardiac information by transmitting, over the network 104, instructions and/or display data to the client device 102. In response, the client device 102 may display the secondary information 414 at the interface 400 based on the instructions and/or display data, as shown in FIG. 4C. In some embodiments, the instructions and/or display data comprises drawing commands for drawing the secondary information 414 on the interface 400.

In the example shown in FIG. 4C, the secondary information 414 comprises detailed information related to the AF episode concerning a particular patient. However, the secondary information may display different cardiac information depending on the information object 406 selected and the parameter 412 associated with the selected information object 406. In some embodiments, when the parameter 412 spatially associated with a cardiac information object 406 is the cardiac imaging category (e.g., parameter 412a), the secondary cardiac information comprises at least one of an image, a video, and an imaging report. For example, the secondary cardiac information can comprise at least one of an echocardiogram image, an echocardiogram video, and an echocardiogram report.

In some embodiments, when the parameter 412 spatially associated with a cardiac information object 406 is the measure of anticoagulation (e.g., parameter 412b), the secondary cardiac information comprises a value of the measure of anticoagulation. The measure of anticoagulation can comprise a prothrombin time. In some embodiments, the measure of anticoagulation can comprise an international normalized ratio of prothrombin time.

In some embodiments, when the parameter 412 spatially associated with a cardiac information object 406 is the cardiac procedure category (e.g., parameter 412c), the secondary cardiac information comprises at least one of an image, a video, and a report related to a cardiac procedure performed on the patient.

In some embodiments, when the parameter 412 spatially associated with a cardiac information object 406 is the encounter (e.g., parameter 412d), the secondary cardiac information comprises a report of the encounter. The report of the encounter can comprise at least one of a report of a patient history and report of a physical examination of the patient.

In some embodiments, when the parameter 412 spatially associated with a cardiac information object 406 is the cardiac rhythm category (e.g., parameter 412e), the secondary cardiac information comprises at least one of an electrocardiogram and a report of an electrocardiogram.

In some embodiments, when the parameter 412 spatially associated with a cardiac information object 406 is the patient portal (e.g., parameter 412f), the secondary cardiac information comprises a patient self-report of at least one of a symptom and a medication use.

According to various embodiments of the subject technology, the method 300 may further comprise facilitating expanding or facilitating compressing a representation of the axis 402 such that the cardiac information objects 406 associated with the portion of the time displayed on the axis 402 are correspondingly expanded or compressed graphically. This may allow a user of the client device 102 to conveniently view more or less information objects 406 in a given area. For example, the user may want to zoom in on a particular area to look at various information objects 406 in greater detail. In another example, the user may want to zoom out to obtain a more general view (and hence a greater number of information objects 406) within a given area of the interface 400. The adjustment module 210 can be configured to facilitate expanding or facilitate compressing the representation of the axis 402 by transmitting, over the network 104, instructions and/or display data to the client device 102. In response, the client device 102 may expand or compress the representation of the axis 402 such that the cardiac information objects 406 associated with the portion of the time displayed on the axis 402 are correspondingly expanded or compressed graphically. In some embodiments, the instructions and/or display data comprises drawing commands for drawing the expanded or compressed axis 402 and/or information objects 406.

In some embodiments, a window may be used for facilitating expansion or facilitating compression of the display of the interface 400. FIG. 4D illustrates an example of such a window 422, in accordance with various embodiments of the subject technology. The window 422 may be displayed by clicking on the tab 408c. The window 422 may also be hidden from view once the tab 408c is clicked on again. In some embodiments, the window 422 comprises a zoomed out view of all the information objects 406 associated with a patient over the entire period of care provided to the patient. However, the window 422 can comprise other suitable views of the information objects 406 over a smaller period of time. The window 422 comprises a left slider 416a and a right slider 416b. The left slider 416a can be moved horizontally relative to the window 422 and represents the leftmost edge of the view as displayed in main area 424 of the interface 400. The right slider 416b can be moved horizontally relative to the window 422 and represents the rightmost edge of the view as displayed in main area 424. Thus, by adjusting the sliders 416a and 416b, a user can zoom in on certain portions of the window 422 or zoom out from the window 422. This view is reflected on the main area 424. The information objects 406 may be correspondingly expanded or compressed graphically.

According to certain embodiments, if a group of information objects 406 are grouped together too closely under a certain view, the group of information objects 406 may be represented by a group symbol. For example, as shown in FIG. 4D, the information object group 418 represents two information objects 406, as indicated by the number “2” displayed on the information object group 418 symbol. If the view as shown in FIG. 4D is zoomed in, for example, the information object group 418 can be separated to show the individual information objects 406 that is part of the information object group 418.

FIG. 4E illustrates an example of a zoomed in view of the interface 400 relative to the view shown in FIG. 4D, in accordance with various embodiments of the subject technology. As compared to the left slider 416a shown in FIG. 4D, the left slider 416a shown in FIG. 4E has been moved toward the right closer to the right slider 416b. Thus, the view of the interface 400 is now zoomed in compared to the view of the interface 400 shown in FIG. 4D. As a result of the zoomed in view, the information objects 406 (e.g., 406e and 406f) that were part of the information object group 418 of FIG. 4D are now visible.

According to various embodiments of the subject technology, the method 300 comprises receiving input from the patient. In some embodiments, the input may be used to generate a patient self-report. For example, the patient input module 208 can be configured to receive the input from the patient and generate the patient self-report based on the input received from the patient. The patient input module 208 may be configured to transmit the patient self-report to the client device 102 for display. In some embodiments, the input from the patient may be used to allow a clinician to diagnose and/or treat the patient. In some embodiments, the input from the patient may be used to indicate to a clinician whether a patient is complying with prescribed therapies. This indication may be represented by an information object 406 (e.g., information object 406c).

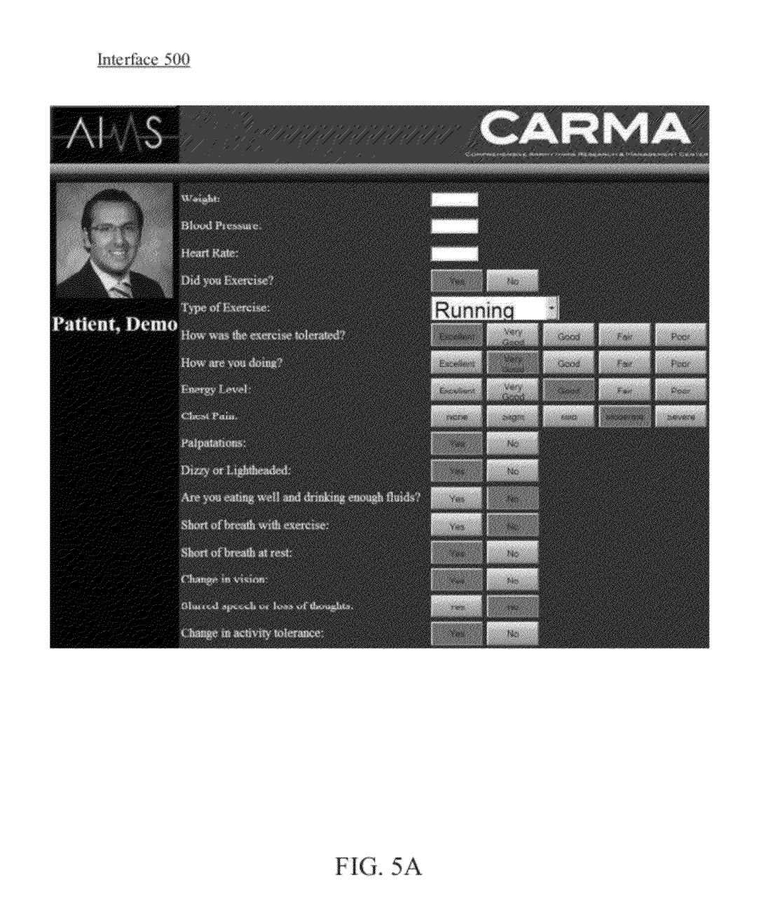

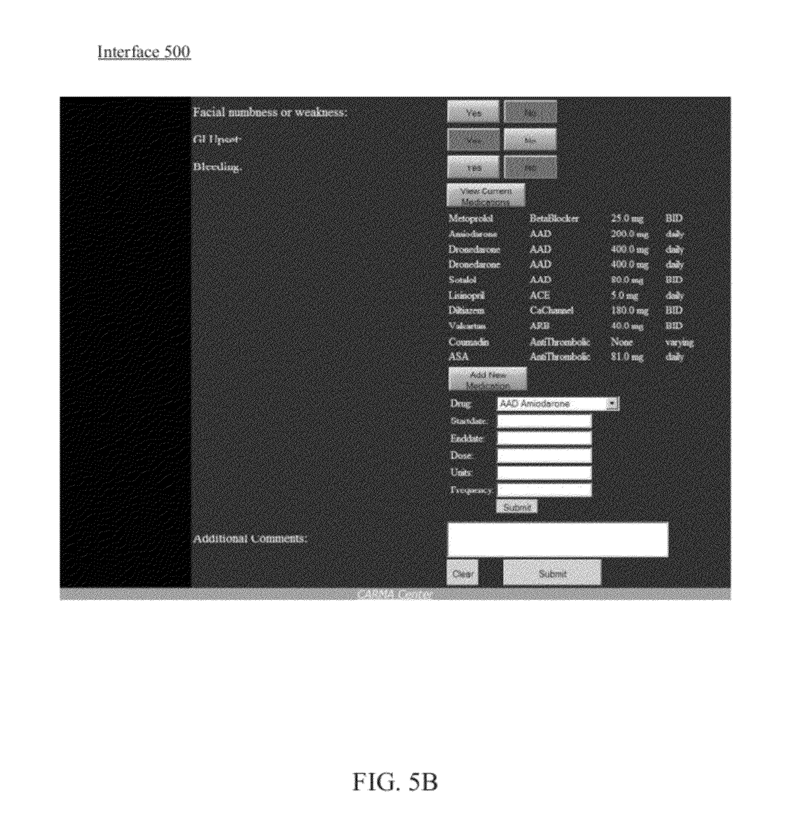

FIGS. 5A and 5B illustrate an example of an interface 500 used for collecting information from a patient, in accordance with various embodiments of the subject technology. The server 106 can be configured to facilitate displaying the interface 500 at the client device 102. In some embodiments, the interface 500 may be displayed once a user clicks on the patient portal parameter 412f. For example, a clinician at a health clinic may use a client device 102 to click on this parameter 412f to display the interface 500. The clinician may allow a patient at the health clinic to fill out the questions presented on the interface 500. The answers to the questions may be used as input to generate the self-report and/or allow the clinician to diagnose and treat the patient.

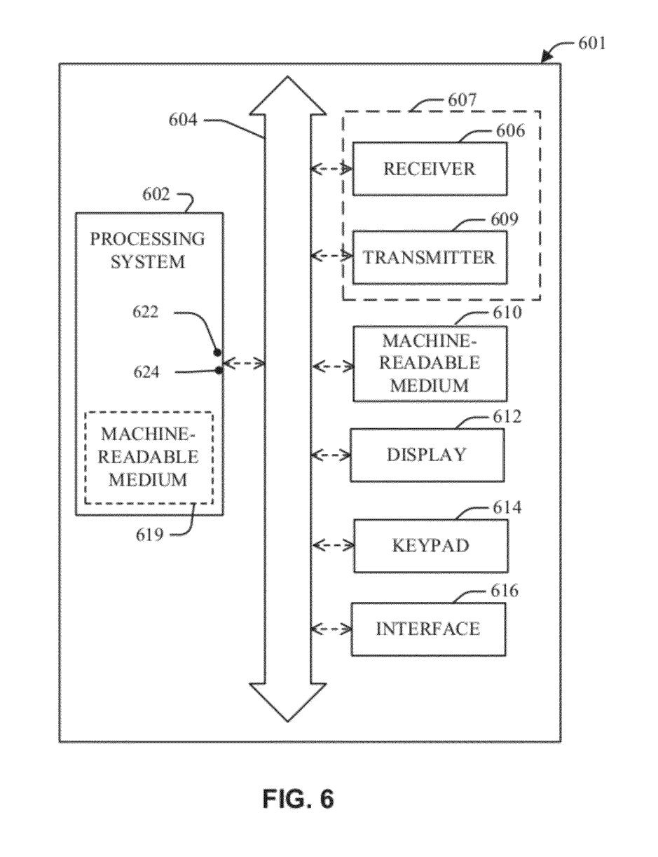

FIG. 6 is a conceptual block diagram illustrating an example of a system, in accordance with various embodiments of the subject technology. A system 601 may be, for example, a client device (e.g., client device 102) or a server (e.g., server 106). The system 601 may include a processing system 602. The processing system 602 is capable of communication with a receiver 606 and a transmitter 609 through a bus 604 or other structures or devices. It should be understood that communication means other than busses can be utilized with the disclosed configurations. The processing system 602 can generate audio, video, multimedia, and/or other types of data to be provided to the transmitter 609 for communication. In addition, audio, video, multimedia, and/or other types of data can be received at the receiver 606, and processed by the processing system 602.

The processing system 602 may include a processor for executing instructions and may further include a machine-readable medium 619, such as a volatile or non-volatile memory, for storing data and/or instructions for software programs. The instructions, which may be stored in a machine-readable medium 610 and/or 619, may be executed by the processing system 602 to control and manage access to the various networks, as well as provide other communication and processing functions. The instructions may also include instructions executed by the processing system 602 for various user interface devices, such as a display 612 and a keypad 614. The processing system 602 may include an input port 622 and an output port 624. Each of the input port 622 and the output port 624 may include one or more ports. The input port 622 and the output port 624 may be the same port (e.g., a bi-directional port) or may be different ports.

The processing system 602 may be implemented using software, hardware, or a combination of both. By way of example, the processing system 602 may be implemented with one or more processors. A processor may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device that can perform calculations or other manipulations of information.

A machine-readable medium can be one or more machine-readable media. Software shall be construed broadly to mean instructions, data, or any combination thereof, whether referred to as software, firmware, middleware, microcode, hardware description language, or otherwise. Instructions may include code (e.g., in source code format, binary code format, executable code format, or any other suitable format of code).

Machine-readable media (e.g., 619) may include storage integrated into a processing system, such as might be the case with an ASIC. Machine-readable media (e.g., 610) may also include storage external to a processing system, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device. Those skilled in the art will recognize how best to implement the described functionality for the processing system 602. According to one aspect of the disclosure, a machine-readable medium is a computer-readable medium encoded or stored with instructions and is a computing element, which defines structural and functional interrelationships between the instructions and the rest of the system, which permit the instructions' functionality to be realized. In one aspect, a machine-readable medium is a non-transitory machine-readable medium, a machine-readable storage medium, or a non-transitory machine-readable storage medium. In one aspect, a computer-readable medium is a non-transitory computer-readable medium, a computer-readable storage medium, or a non-transitory computer-readable storage medium. Instructions may be executable, for example, by a client device or server or by a processing system of a client device or server. Instructions can be, for example, a computer program including code.

An interface 616 may be any type of interface and may reside between any of the components shown in FIG. 6. An interface 616 may also be, for example, an interface to the outside world (e.g., an Internet network interface). A transceiver block 607 may represent one or more transceivers, and each transceiver may include a receiver 606 and a transmitter 609. A functionality implemented in a processing system 602 may be implemented in a portion of a receiver 606, a portion of a transmitter 609, a portion of a machine-readable medium 610, a portion of a display 612, a portion of a keypad 614, or a portion of an interface 616, and vice versa.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “right,” “left” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A method, for managing cardiac arrhythmia information concerning a patient, comprising:
facilitating, by a processor, displaying cardiac information objects graphically at an interface having at least two axes;
wherein time is displayed on a first axis of the at least two axes;
wherein parameters of the cardiac information objects are displayed on a second axis of the at least two axes;
wherein each cardiac information object is spatially associated with at least a portion of the time displayed and a parameter displayed;
wherein the displayed parameters respectively comprise a cardiac imaging category, a measure of anticoagulation, a cardiac procedure category, an encounter of the patient with a clinician, a cardiac rhythm category, a patient portal, and a cardiac therapy category;
receiving, by a processor, an inquiry input associated with a first of the cardiac information objects; and
facilitating displaying, in response to the inquiry input and by a processor, secondary cardiac information corresponding to the parameter spatially associated with the first of the cardiac information objects;
wherein, when the parameter spatially associated with the first of the cardiac information objects is the cardiac imaging category, the secondary cardiac information comprises at least one of an image, a video, and an imaging report;
wherein, when the parameter spatially associated with the first of the cardiac information objects is the measure of anticoagulation, the secondary cardiac information comprises a value of the measure of anticoagulation;
wherein, when the parameter spatially associated with the first of the cardiac information objects is the cardiac procedure category, the secondary cardiac information comprises at least one of an image, a video, and a report related to a cardiac procedure performed on the patient;
wherein, when the parameter spatially associated with the first of the cardiac information objects is the encounter, the secondary cardiac information comprises a report of the encounter;
wherein, when the parameter spatially associated with the first of the cardiac information objects is the cardiac rhythm category, the secondary cardiac information comprises at least one of an electrocardiogram and a report of an electrocardiogram;
wherein, when the parameter spatially associated with the first of the cardiac information objects is the patient portal, the secondary cardiac information comprises a patient self-report of at least one of a symptom and a medication use.

2. The method of claim 1, wherein the cardiac information object spatially associated with the cardiac therapy category parameter comprises (a) a first edge indicating a time of onset of a prescribed therapy, and (b) a second edge indicating (i) a time of cessation of the prescribed therapy, or (ii) a present time.

3. The method of claim 2, wherein the prescribed therapy comprises administration of a medication, and wherein at least one of a width and a color of the cardiac information objects associated with the prescribed therapy varies with a dosage of the medication.

4. The method of claim 1, wherein, when the parameter spatially associated with the first of the cardiac information objects is the cardiac imaging category, the secondary cardiac information comprises at least one of an echocardiogram image, an echocardiogram video, and an echocardiogram report.

5. The method of claim 1, wherein the cardiac information objects are displayed discretely along the first axis.

6. The method of claim 1, further comprising receiving input from the patient, the input received from the patient configured to generate the patient self-report.

7. The method of claim 6, further comprising generating the patient self-report based on the input received from the patient.

8. The method of claim 1, wherein the measure of anticoagulation comprises a prothrombin time.

9. The method of claim 1, wherein the measure of anticoagulation comprises an international normalized ratio of prothrombin time.

10. The method of claim 1, wherein the report of the encounter comprises at least one of a report of a patient history and report of a physical examination of the patient.

11. The method of claim 1, wherein the first axis and the second axis are orthogonal to one another.

12. The method of claim 1, further comprising facilitating expanding or facilitating compressing a representation of the first axis such that the cardiac information objects associated with the at least the portion of the time displayed on the first axis are correspondingly expanded or compressed graphically.

13. A non-transitory machine-readable medium encoded with instructions executable by a processing system to perform a method for managing cardiac arrhythmia information concerning a patient, the instructions comprising code for:
facilitating, by a processor, displaying cardiac information objects graphically at an interface having at least two axes;
wherein time is displayed on a first axis of the at least two axes;
wherein parameters of the cardiac information objects are displayed on a second axis of the at least two axes;
wherein each cardiac information object is spatially associated with at least a portion of the time displayed and a parameter displayed;
wherein the displayed parameters respectively comprise a cardiac imaging category, a measure of anticoagulation, a cardiac procedure category, an encounter of the patient with a clinician, a cardiac rhythm category, a patient portal, and a cardiac therapy category;
receiving, by a processor, an inquiry input associated with a first of the cardiac information objects; and
facilitating displaying, in response to the inquiry input and by a processor, secondary cardiac information corresponding to the parameter spatially associated with the first of the cardiac information objects;
wherein, when the parameter spatially associated with the first of the cardiac information objects is the cardiac imaging category, the secondary cardiac information comprises at least one of an image, a video, and an imaging report;
wherein, when the parameter spatially associated with the first of the cardiac information objects is the measure of anticoagulation, the secondary cardiac information comprises a value of the measure of anticoagulation;
wherein, when the parameter spatially associated with the first of the cardiac information objects is the cardiac procedure category, the secondary cardiac information comprises at least one of an image, a video, and a report related to a cardiac procedure performed on the patient;
wherein, when the parameter spatially associated with the first of the cardiac information objects is the encounter, the secondary cardiac information comprises a report of the encounter;
wherein, when the parameter spatially associated with the first of the cardiac information objects is the cardiac rhythm category, the secondary cardiac information comprises at least one of an electrocardiogram and a report of an electrocardiogram;
wherein, when the parameter spatially associated with the first of the cardiac information objects is the patient portal, the secondary cardiac information comprises a patient self-report of at least one of a symptom and a medication use.

14. The machine-readable medium of claim 13, wherein the instructions further comprise code for receiving input from the patient, the input received from the patient configured to generate the patient self-report.

15. The machine-readable medium of claim 13, wherein the instructions further comprise code for facilitating expanding or facilitating compressing a representation of the first axis such that the cardiac information objects associated with the at least the portion of the time displayed on the first axis are correspondingly expanded or compressed graphically.

16. A computing machine comprising the machine-readable medium of claim 13.

17. A system, for managing cardiac arrhythmia information concerning a patient, comprising:
a first display module configured to facilitate, by a processor, displaying cardiac information objects graphically at an interface having at least two axes;
wherein time is displayed on a first axis of the at least two axes;
wherein parameters of the cardiac information objects are displayed on a second axis of the at least two axes;
wherein each cardiac information object is spatially associated with at least a portion of the time displayed and a parameter displayed;
wherein the displayed parameters respectively comprise a cardiac imaging category, a measure of anticoagulation, a cardiac procedure category, an encounter of the patient with a clinician, a cardiac rhythm category, a patient portal, and a cardiac therapy category;
a user input module configured to receive, by a processor, an inquiry input associated with a first of the cardiac information objects; and
a second display module configured to facilitate displaying, in response to the inquiry input and by a processor, secondary cardiac information corresponding to the parameter spatially associated with the first of the cardiac information objects;
wherein, when the parameter spatially associated with the first of the cardiac information objects is the cardiac imaging category, the secondary cardiac information comprises at least one of an image, a video, and an imaging report;
wherein, when the parameter spatially associated with the first of the cardiac information objects is the measure of anticoagulation, the secondary cardiac information comprises a value of the measure of anticoagulation;
wherein, when the parameter spatially associated with the first of the cardiac information objects is the cardiac procedure category, the secondary cardiac information comprises at least one of an image, a video, and a report related to a cardiac procedure performed on the patient;
wherein, when the parameter spatially associated with the first of the cardiac information objects is the encounter, the secondary cardiac information comprises a report of the encounter;
wherein, when the parameter spatially associated with the first of the cardiac information objects is the cardiac rhythm category, the secondary cardiac information comprises at least one of an electrocardiogram and a report of an electrocardiogram;
wherein, when the parameter spatially associated with the first of the cardiac information objects is the patient portal, the secondary cardiac information comprises a patient self-report of at least one of a symptom and a medication use.

18. The system of claim 17, further comprising a patient input module configured to receive input from the patient, the input received from the patient configured to generate the patient self-report.

19. The system of claim 18, wherein the patient input module is configured to generate the patient self-report based on the input received from the patient.

20. The system of claim 17, further comprising an adjustment module configured to facilitate expanding or facilitate compressing a representation of the first axis such that the cardiac information objects associated with the at least the portion of the time displayed on the first axis are correspondingly expanded or compressed graphically.
